(19) United States
(12) Plant Patent Application Publication
(10) Pub. No.: US 2013/0318669 P1
(43) Pub. Date: Nov. 28, 2013

(54) Campanula plant named 'L. 10.061'

(50) ;

(71) Applicant: Gert Kim Jensen, Marslev (DK)

(72) Inventor: Gert Kim Jensen, Marslev (DK)

(73) Assignee: ROSA-DANICA A/S

(21) Appl. No.: 13/506,926

(22) Filed: May 25, 2012

(51) Int. Cl. A01H 5/00

(52) U.S. Cl. PLT/414

(57) ABSTRACT

A new and distinct cultivar of Campanula plant named ‘L.10.061’, characterized by its compact upright plant habit; dense and bushy plant form; vigorous growth habit; and large upright, white flowers.

### LATIN NAME OF THE GENUS AND SPECIES OF THE CLAIMED PLANT

Campanula portenschlagiana Schult.

### VARIETY DENOMINATION

‘L.10.061’

### BACKGROUND OF THE INVENTION

The present invention relates to a new and distinct cultivar of Campanula plant, botanically known as Campanula portenschlagiana Schult., commonly known as Dalmatian Bellflower and hereinafter referred to by the cultivar name ‘L.10.061’.

The new Campanula cultivar is a product of a planned breeding program conducted by the inventor, Gert Jensen, in Marslev, Denmark.

The new Campanula originated from a selfing cross made in 2010 by the Inventor between plants of the Campanula portenschlagiana Schult. variety ‘A.09.09’ (breeder reference, unpatented). The Inventor selected the new Campanula cultivar from the progeny of the above crossing in 2011 on the basis of its compact, upright growth, with low need for retardation and fungicides and distinctive flowering habit.

Asexual reproduction of the new Campanula cultivar has demonstrated that the combination of characteristics as herein disclosed for the new cultivar are firmly fixed and retained through successive generations of asexual reproduction. The new cultivar reproduces true to type.

### BRIEF SUMMARY OF THE INVENTION

The following traits have been repeatedly observed and are determined to be unique characteristics of ‘L.10.061’, which in combination distinguish this Campanula as a new and distinct cultivar:

1. Larger, white, single bell flowers;

2. High numbers of flowers per plant;

3. Dense and bushy plant form, mainly due to more upright stems;

4. Upright plant habit;

5. Less need for growth retardant.

Plants of the cultivar ‘L 10.018’ can be compared to plants of the Campanula portenschlagiana Schult. cultivar ‘White Get Mee’ (breeder reference, unpatented). However, in side-by-side comparisons conducted by the Inventor in Marslev, Denmark, plants of the cultivar ‘L.10.061’ and the cultivar ‘White Get Mee’ differ in the following characteristics, it must however be mentioned that the new cultivar plant examined has not been treated with growth retardant.

1. Plants of the new Campanula have white, single bell, upright flowers.

2. Plants of the new Campanula have more upright growth than plants of the cultivar ‘White Get Mee’.

3. Plants of the new Campanula do not need growth retardant or cooling treatment.

4. Plants of the new Campanula have smaller leaves and thus less conspicuous foliage when flowering.

5. Plants of the new Campanula have more flowers per plant than the plants of the cultivar ‘B.09.01’.

6. Plants of the new Campanula have larger flowers than the plants of the cultivar ‘White Get Mee’.

7. Plants of the new Campanula appear to be less susceptible to grey mold.

### BRIEF DESCRIPTION OF THE PHOTOGRAPHS

The accompanying photographs illustrate the overall appearance of the new Campanula ‘L.10.061’ showing the colors as true as is reasonably possible with colored reproductions of this type. Colors in the photographs may differ slightly from the color value cited in the detailed botanical description, which accurately describe the color of ‘L.10.061’.

FIG. 1 shows a side perspective view of a typical flowering plant of ‘L.10.061’ compared to a typical flowering plant of ‘White Get Mee’ grown in 10.5 cm pots.

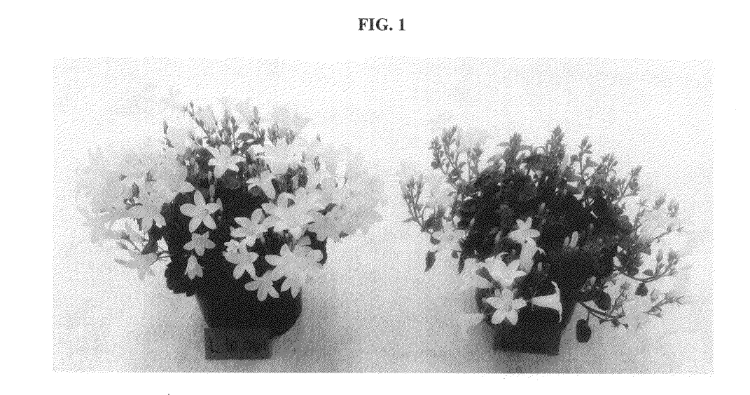

FIG. 2 shows a close-up view of typical single flowers of ‘L.10.061’ along with typical single flowers of ‘White Get Mee’.

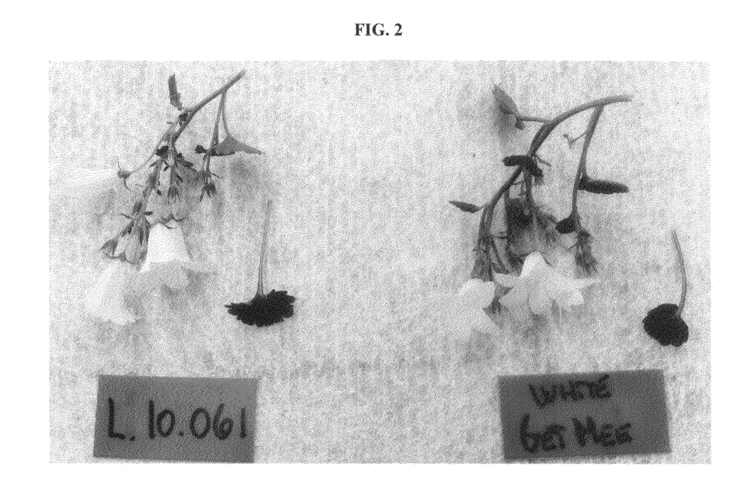

### DETAILED BOTANICAL DESCRIPTION

The new Campanula ‘L.10.061’ has not been observed under all possible environmental conditions. The phenotype of the new cultivar may vary with variations in environment such as temperature, light intensity, and day length without any change in the genotype of the plant.

In the following description, color references are made to The Royal Horticultural Society Colour Chart, 4^{th} edition, where general terms of ordinary dictionary significance are used. Plants were grown under greenhouse conditions with an average day temperature of 18-20° C., an average night temperature of 16° C., ambient (80 watt) light level, and 7 weeks of long day treatments (18 hrs). Plants used for this description were grown for about 14 weeks after cutting.
Botanical classification: Campanula portenschlagiana Schult cultivar ‘L.10.061’.
Parentage:
   Female parent.—Campanula portenschlagiana Schult, ‘A.09.09’.
   Male parent.—Campanula portenschlagiana Schult, ‘A.09.09’.
Propagation:
   Type cutting.—Terminal vegetative cuttings from stock plants maintained at short days.
   Time to initiate roots.—About 10 to 14 days at 18 to 21 C. in tunnels in a greenhouse.
   Root description.—Fine, well branched.
Plant description:
   Form.—Perennial plant with an upright globular plant habit. Campanulate flowers in racemes. Freely branching with lateral branches forming at every node; dense and bushy. Freely branching, somewhat brittle stems.
   Crop time.—After rooting, about 14 weeks in long days are required to produce finished flowering plants in 11 cm pots.
   Plant height (soil level to top of plant plane).—About 14 cm.
   Plant diameter.—18 cm.
   Vigor.—Vigorous growth rate.
Foliage description: Leaves alternate, single, dentate, palmate (actinodromus), not very conspicuous venation.
   Basal leaves.—Length: 17-19 mm. Width: About 24 mm. Shape: Cordate. Apex: acuminate. Base: Cordate. Margin: broadly dentate. Texture: smooth, glabrous, dull. Color: Young foliage, upper and lower surfaces respectively: RHS 137B, green and RHS N138C. Mature foliage, upper and lower surfaces respectively: RHS 137A, and N138C, green. Venation, RHS 138C.
   Petioles.—on basal leaves: 4 cm on apical leaves 5-31 mm.
Flower description:
   Flower arrangement and shape.—Single flowers in racemes; Violet campanulate flowers with small star shaped calyx.
   Natural flowering season.—Continuous throughout the spring and summer in Denmark. Season can be extended by long day treatments. 7 weeks (14 hour) are needed for flower induction under greenhouse conditions.
   Flower longevity on the plant.—Longevity (9 to 14 days) of individual flowers is highly dependent on temperature and light conditions. Flowers persistent.
   Inflorescence size.—Length: About 6 cm.
   Flower buds.—Color: RHS 155C, white with ridges Shape: Oblong Texture: with ridges Length: up to 6 mm long Width: up to 5 mm before anthesis.
   Flowers.—Attitude: Upright, Length (height): About 19 mm. Diameter: About 28 mm. Shape: Elliptic, rounded, reflexing.
   Petal lobes.—Length: 10 mm Width: 8 mm.
   Corolla tube.—Depth: 11 mm Width: 10 mm Color: Upper and lower surfaces, RHS 155D, withering to RHS 156A.
Pistil and stigma: conspicuously tripartite, same color as petals
Peduncle:
   Strength.—Moderately strong.
   Length.—About 20 mm.
   Diameter.—About 0.5 mm.
   Color.—RHS 144C yellow-green.
Weather tolerance: Plants of the new Campanula have not been tested for tolerance to drought, rain and wind, low temperature.
Disease resistance: Plants of the new claimed variety have shown good resistance to grey mould.

What is claimed is:

1. A new and distinct cultivar of Campanula plant named ‘L.10.061’, as illustrated and described herein.
